(19) United States
(12) Patent Application Publication
(10) Pub. No.: US 2016/0022336 A1
(43) Pub. Date: Jan. 28, 2016

(54) Apparatus for Humeral Fracture Repair

(71) Applicant: Edward Richard BATEMAN, North Gosford, New South Wales (AU)

(72) Inventor: Edward Richard Bateman, New South Wales (AU)

(21) Appl. No.: 14/773,532

(22) PCT Filed: Mar. 5, 2014
(86) PCT No.: PCT/AU2014/000205

§ 371 Date: Sep. 8, 2015

(30) Foreign Application Priority Data
Mar. 8, 2013 (AU) 2013900819

(51) Int. Cl. A61B 17/80

(52) U.S. Cl. CPC A61B 17/8061; A61B 17/8033

(57) ABSTRACT

Apparatus for repair of a proximal humeral fracture is disclosed including a fixation plate adapted to be located at an external surface of the proximal humerus, a support element adapted to be implanted in the proximal humerus; and one or more connection elements adapted to connect the fixation plate to the support element.

### CROSS-REFERENCE TO RELATED APPLICATIONS

The present application claims priority from Australian Provisional Patent Application No 2013900819 filed on 8 Mar. 2013, the contents of which are incorporated herein by reference.

### TECHNICAL FIELD

The present disclosure relates to apparatus for repair of fractured bones including proximal humeral fractures.

### BACKGROUND

Fractures of the proximal humerus are a common injury to the shoulder, occurring more frequently for older individuals and those suffering from osteoporosis, and affecting females over males by a ratio of about 3 to 1.

While most minor proximal humeral fractures can be treated non-operatively, more severe cases require surgical treatment. For example, it is common to treat major or unstable fractures using fixation plates or intramedullary nails. When fixation plates are used, a fractured portion of the humeral head is realigned and the proximal end of the humerus is stabilized by multiple screws. The screws extend through the fixation plate, which is located in a lateral position external to the bone. The screws secure the plate to the humerus and project into the bone, stabilizing the bone adjacent the fracture during healing. When intramedullary nails are used, after realignment, the nail is placed through the medullary cavity of the bone such that it extends across the fracture and is locked in position at its ends using bolts or screws.

Using known techniques, however, it has been found that about 30% of proximal humeral fracture repairs fail. Failure usually occurs due to collapse of the proximal humerus into the varus position, which can cause significant pain and stiffness to the patient.

Any discussion of documents, acts, materials, devices, articles or the like which has been included in the present specification is not to be taken as an admission that any or all of these matters form part of the prior art 1:4se or were common general knowledge in the field relevant to the present disclosure as it existed before the priority date of each claim of this application.

### SUMMARY

According to one aspect of the present disclosure, there is provided apparatus for repair of a proximal humeral fracture comprising:

a fixation plate adapted to locate at an external surface of the proximal humerus,

a support element adapted to be implanted in the proximal humerus; and

one or more connection elements adapted to connect the fixation plate to the support element.

According to another aspect of the present disclosure, there is provided a support element for repair of a proximal humeral fracture, the support element adapted to be implanted in the proximal humerus and configured to be attached to connection elements extending into the humerus from a fixation plate located at an external surface of the proximal humerus.

According to another aspect of the present disclosure, there is provided a method for repair of a proximal humeral fracture comprising:

implanting a support element in a proximal humerus;

locating a fixation plate at a surface of the proximal humerus; and

connecting one or more connection elements between the fixation plate and the support element.

Aspects and embodiments disclosed herein may provide improved means for repair of a fractured proximal humerus. While the fixation plate located to the surface of the humerus may provide support to the bone during the healing process, by implanting a support element at least partially in the humerus, which can be stabilised at least partially through its connection to the fixation plate, the support for the bone during the healing process can be greater and the proximal humerus can be less prone to collapsing into the varus position.

The support element may be a support plate. The support element may be adapted to locate against the internal surface of the medial bone shaft. The support element may be an intramedullary plate in some embodiments. Two opposing surfaces of the intramedullary plate may be substantially flat or may be curved, e.g. to follow internal surface contours of the medial shaft of the proximal humerus. Nevertheless, the support element may take a variety of different shapes or configurations that are capable of attachment to connection elements to connect the support element to the fixation plate. For example, the support element may be an intramedullary rod, nail or otherwise.

The support element may be formed of a variety of different materials. For example, the support element may comprise metal such as stainless steel, or titanium, etc., or plastic such as polyetheretherketone (PEEK), etc. Where a plastic such as PEEK is used, the support element may further comprise radiopaque material or radiopaque markers for easier visualisation under x-ray analysis, etc.

The support element, e.g. the intramedullary plate, may be smaller than the fixation plate in some embodiments. Therefore, the support element may have a smaller length, thickness and/or width than the fixation plate. By being smaller, the support element may have a profile that is more suitable for implantation. Nonetheless, in one embodiment, the support element may have a width that is greater than the width of the fixation plate.

The fixation plate may be wider at a proximal end or proximal portion than at a distal end or distal portion. The width of the fixation plate may progressively increases towards the proximal end of the fixation plate from a central region of the fixation plate. The proximal portion of the fixation plate may be asymmetrically arranged such as to define a proximal-posterior wing of the fixation plate.

The support element may be wider at a proximal end or proximal portion than at a distal end or distal portion. The width of the support element may taper towards the distal end of the support element from a central region of the support element. The proximal portion of the support element may be defined by a lateral bend in the support element. The bend may be between about 5 degrees and about 20 degrees, e.g., about 10 degrees. The proximal portion of the support element may be asymmetrically arranged such as to define a proximal-posterior wing of the support element. The support element may be “L”-shaped. The posterior wing and/or “L”-shape may provide for improved support in consideration of retroversion of the humeral head. A left-side and a right-side version of the support element and/or fixation plate may be provided. One or more openings to receive connection elements may be provided in the proximal portion of the fixation plate and/or support element. The proximal-posterior wing of the support element may be connected to the proximal-posterior wing of the fixation plate by one or more connection elements. The proximal portion of the support element may comprise an elongate opening, the elongate opening extending in a direction perpendicular to the axis of elongation of the support element.

The connection elements may comprise bone screws or other elongate fixation elements such as rods or bolts. While in some embodiments, all elongate fixation elements such as bone screws may connect the fixation plate to the support element, in other embodiments, additional fixation elements may be provided that do not connect the fixation plate to the support element. The additional fixation elements may serve only to fix the fixation plate to the external surface of the proximal humerus, or serve only to fix the support element to an internal surface of the proximal humerus, for example. In one embodiment, one or more connecting screws are connected between the fixation plate and the support element, one or more fixing screws fix the fixation plate to the external surface of the proximal humerus, and one or more fixing screws fix the support element to the proximal humerus, e.g., to the internal surface of the medial shaft. At one or both of the fixation plate and the support element, fixing screws may be located at least distally of the connecting screws. Nonetheless, at one or both of the fixation plate and the support element, fixing screws may also be located proximally of the connecting screws.

The fixation plate may comprise one or more openings, e.g. holes or bores, through which respective connection elements extend to connect to the fixation plate. Similarly, the support element may comprise one or more openings, e.g. holes or bores, through which respective connection elements extend to connect to the support element. When the connection elements are screws, the openings may or may not comprise screw threads. The openings in the fixation plate and/or support element may be round openings, sized substantially in accordance with the screw diameter, or elongated openings, e.g. oblong, ovate, elliptical or rectangular openings, etc.

One or more elongate openings may be provided in the fixation plate and/or support element such that a surgeon installing the apparatus has greater flexibility/freedom to extend a connecting element through elongate opening, e.g., after the fixation plate and support element are fixed in axially aligned positions.

In one embodiment, to install the apparatus, the support element, e.g. intramedullary plate, is implanted in the bone prior to fixing of the fixation plate to the bone. The support element can be implanted while the elbow is maintained bent to approximately 90 degrees. The support element can be lined up for insertion in a position substantially at right angles to the forearm. A holding instrument can be used to maintain the support element in its final position while the support element is fixed to e.g. the internal surface of the medial shaft of the bone.

Once the support element is implanted, the fixation plate can be fixed to the external surface of the bone, with its axis of elongation aligned substantially parallel to the axis of elongation of the support element. The connecting elements can then be connected between the fixation plate and the support element. Nonetheless, alternative approaches to installation may be taken. As one example, one or more connection elements may be connected between the fixation plate and the support element prior to fixing of the fixation plate to the external surface of the bone.

Throughout this specification the word “comprise”, or variations such as “comprises” or “comprising”, will be understood to imply the inclusion of a stated element, integer or step, or group of elements, integers or steps, but not the exclusion of any other element, integer or step, or group of elements, integers or steps.

### BRIEF DESCRIPTION OF DRAWINGS

By way of example only, embodiments are now described with reference to the accompanying drawings, in which:

FIG. 1 shows a cross-sectional view of proximal humeral fracture repair apparatus according to an embodiment of the present disclosure;

FIG. 2 shows a plan view of a lateral plate of the apparatus of FIG. 1;

FIG. 3 shows a plan view of an intramedullary plate of the apparatus of FIG. 1;

FIG. 4 shows an oblique view of an intramedullary plate according to another embodiment of the present disclosure;

FIG. 5a shows an anterior-lateral view of proximal humeral fracture repair apparatus according to an embodiment of the present disclosure, the apparatus including the intramedullary plate of FIG. 4;

FIG. 5b shows a medial view of the apparatus of FIG. 5a;

FIG. 5c shows an anterior view of the apparatus of FIG. 5a;

FIG. 6 shows an oblique view of an intramedullary plate according to yet another embodiment of the present disclosure;

FIG. 7a shows an anterior-lateral view of proximal humeral fracture repair apparatus according to an embodiment of the present disclosure, the apparatus including the intramedullary plate of FIG. 6;

FIG. 7b shows a medial view of the apparatus of FIG. 7a;

FIG. 7c shows an anterior view of the apparatus of FIG. 7a; and

FIG. 8 shows an oblique view of an intramedullary plate according to another embodiment of the present disclosure.

### DESCRIPTION OF EMBODIMENTS

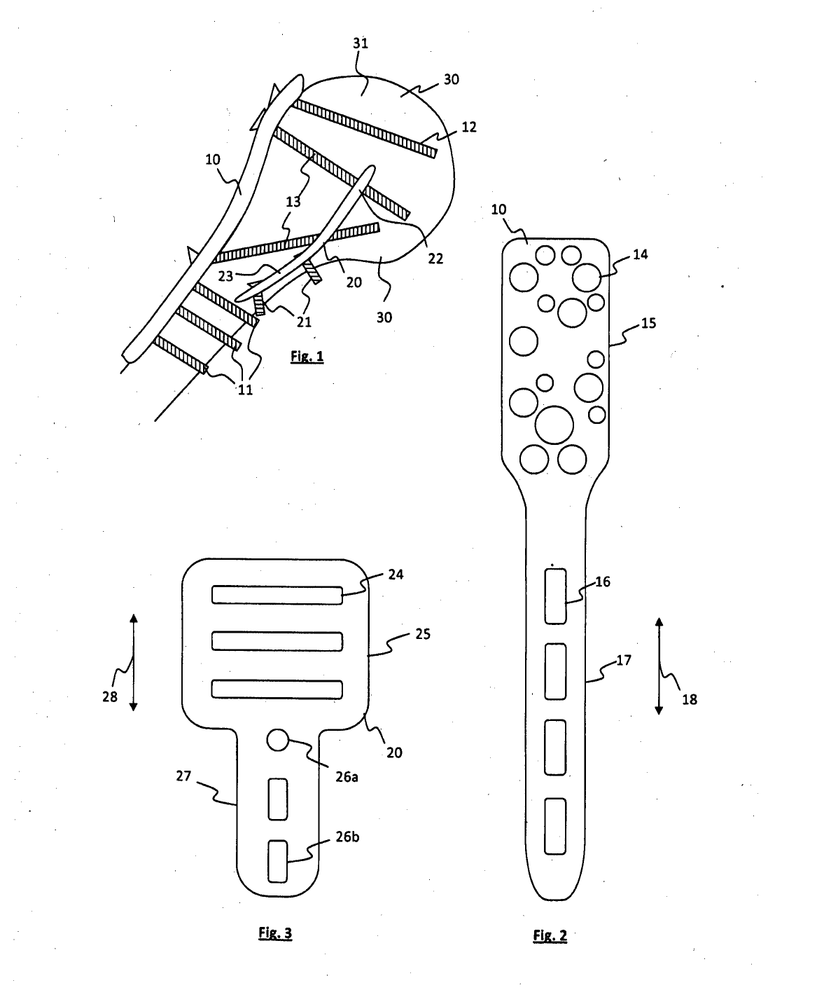

A cross-sectional view of proximal humeral fracture repair apparatus according to an embodiment of the present disclosure is shown in FIG. 1. The apparatus includes a fixation plate, in particular a lateral plate 10, located at a lateral surface of the proximal humerus 30. The apparatus also includes a support element, in particular an intramedullary plate 20, that is implanted in the proximal humerus 30. The axes of elongation of the lateral and intramedullary plates 10, 20 are substantially parallel to each other and to the bone axis in this embodiment.

The intramedullary plate 20 can be positioned against the internal surface of the medial shaft. The lateral plate 10 can be positioned in accordance with the positioning of known lateral plates, used in standard techniques for repairing proximal humeral fractures. For example, the lateral plate 10 can be positioned posterior to the biceps groove and distal to the greater tuberosity 31 of the humerus.

The apparatus includes a plurality of bone screws 11, 12, 21 that fix the lateral and intramedullary plates 10, 20 in position with respect to the bone 30 (“fixing screws”). The apparatus further includes one or more bones screws 13 that connect the lateral and intramedullary plates 10, 20 together (“connecting screws”). In this embodiment, one or more distal fixing screws 11 fix a distal portion of the lateral plate 10 to the bone, one or more proximal fixing screws 12 fix a proximal portion of the lateral plate 10 to the bone, and one or more connecting screws 13, which are located proximally of the distal bone screws 11 and distally of the proximal bone screws 12, at least in this embodiment, extend through the bone to connect the plates 10, 20 together. The connecting screws 13 extend from the lateral plate 10 to connection positions of the intramedullary plate 20 that are provided in substantially a proximal half 22 of the intramedullary plate 20. The apparatus further comprises one or more distal fixing screws 21 that fix substantially a distal half 23 of the intramedullary plate 20 to the bone. Accordingly, at least in this embodiment, the fixing screws 21 for, the intramedullary plate 20 are located distally of positions where the connecting screws 13 connect to the intramedullary plate 20.

Nevertheless, a variety of different fixing and connecting screw configurations may be employed in embodiments according to the present disclosure. For example, the intermediate plate may include fixing screws at its proximal end, similar to the lateral plate 10 as shown in FIG. 1. On the other hand, as another example, the lateral plate may include no fixing screws at its proximal end, similar to the intramedullary plate 20 as shown in FIG. 1.

In accordance with standard bone plating techniques, the lateral and intramedullary plates 10, 20 include a plurality of openings (e.g. holes, bores, etc.) through which the respective screws extend. In relation to the fixing screws, 11, 12, 21, the respective openings in the lateral and intramedullary plates 10, 20 are sized so that a head of each fixing screw can press against a surrounding portion of the opening, enabling plate fixation. In relation to the connecting screws 13, the respective openings in the lateral plate are also sized so that a head of each connecting screw can press against a surrounding portion of the opening. However, connection with the intramedullary plate 20 is achieved by the connecting screws passing through respective openings in the intramedullary plate 20.

A plan view of the lateral plate 10 is shown in FIG. 2. The lateral plate 10 may be configured in accordance with known lateral plates 10, e.g. to the extent that it has a size and a sufficient number and appropriate positioning of openings to achieve screw arrangements such as that shown in FIG. 1, for example. Alternatively, the lateral plate 10 may have a configuration that is tailored for use specifically in conjunction with an intramedullary plate in accordance with the present disclosure. As shown in FIG. 2, the lateral plate 10 can include a plurality of substantially round openings 14 at a proximal portion 15, and a plurality of elongate (e.g. substantially rectangular or oblong) openings 16 at a distal portion 17. The elongate openings 16 are elongated in substantially the axial direction of the lateral plate 10, which is indicated by arrow 18 in FIG. 2. The proximal portion 15 of the lateral plate 10 can have a width that is larger than the width of the distal portion 17 of the lateral plate 10.

A plan view of the intramedullary plate 20 is shown in FIG. 3. The intramedullary plate includes a plurality of elongate openings 24 at a proximal portion 25, and a plurality of substantially round and/or elongate openings 26a, 26b at a distal portion 27. The elongate openings 24 at the proximal portion 25 are elongate in a direction substantially perpendicular to the axial direction of the intramedullary plate 20, which axial direction is indicated by arrow 28 in FIG. 3. The elongate openings 26b at the distal portion 27 are elongate in substantially the axial direction 28 of the intramedullary plate 20. The proximal portion 25 of the intramedullary plate 20 can have a width that is larger than the width of the distal portion 27 of the intramedullary plate 20. The length of the intramedullary plate 20 is shorter than the length of the lateral plate 10. The intramedullary plate 20 can have a width that is greater than a maximum width of the lateral plate 10 in some embodiments.

In the embodiment shown in FIG. 1, the connecting screws 13 connect through the elongate openings 24 at the distal end portion of the intramedullary plate 20. Since the openings 24 are elongate, some degree of freedom is available, at least in a direction perpendicular to the axial direction 28, to position the connecting screws 13 therethrough. This can make deployment of the apparatus more straightforward, particularly under x-ray, and allow the apparatus to adapt to different bone shapes and sizes, for example. Similarly, the configuration of openings in the lateral plate 10 in this embodiment allows variable angle screw fixation. In general, this can ensure that a surgeon can connect bone screws between the lateral and intramedullary plates 10, 20 while maintaining the plates 10, 20 axially parallel.

In one embodiment, to install the apparatus, the intramedullary plate 20 is implanted in the bone prior to fixing of the lateral plate 10 to an external surface of the bone. The intramedullary plate 20 can be implanted while the elbow is maintained bent to approximately 90 degrees. The intramedullary plate 20 is lined up for insertion in a position substantially at right angles to the forearm. A holding instrument can be used to maintain the intramedullary plate 20 in its desired implantation position while the fixing screws 21 are inserted through the openings 26a, 26b at the distal portion 27 of the plate 20, fixing the intramedullary plate 20 to the internal surface of the medial shaft of the bone.

Once the intramedullary plate 20 in implanted, fixing screws 11, 12 are inserted through the openings 14, 16 at the proximal and distal portions 15, 17 of the lateral plate 10, fixing the lateral plate 10 to the external surface of the bone such that the axial direction 18 (axis of elongation) of the lateral plate 10 is substantially parallel to the axial direction 28 (axis of elongation) of the intramedullary plate 20. The connecting screws 13 can then be inserted through openings 14, 16 in the lateral plate and through openings 24 in the intramedullary plate.

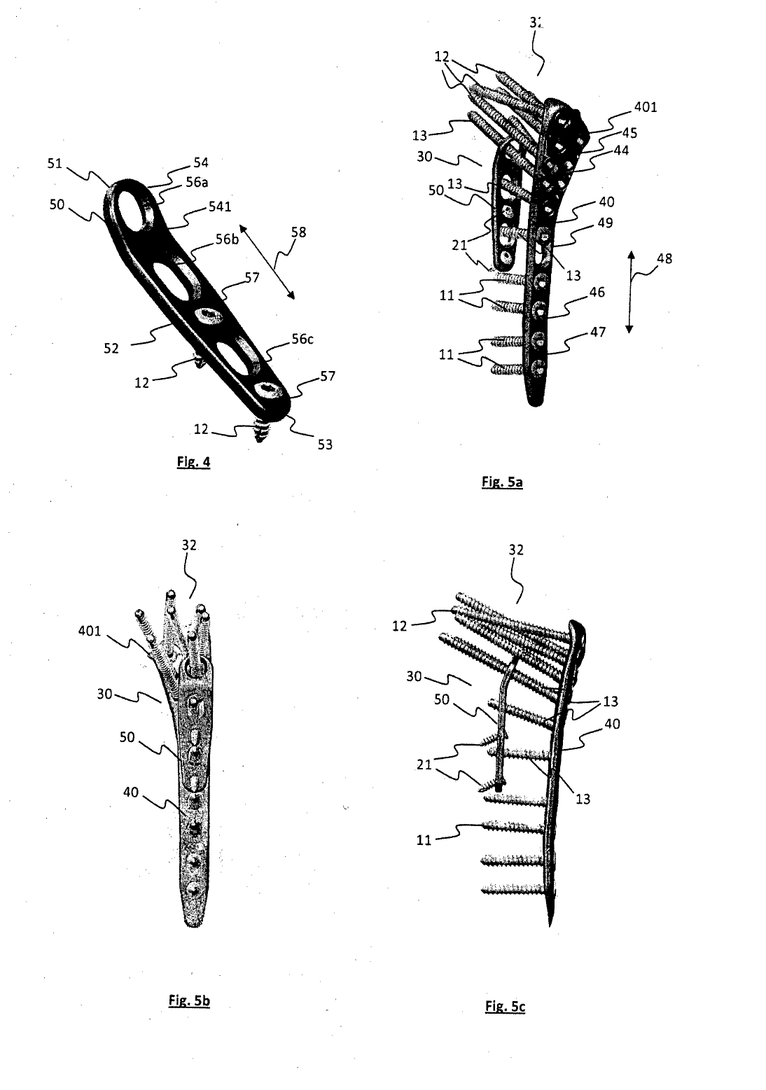

Proximal humeral fracture repair apparatus according to another embodiment of the present disclosure is represented in FIGS. 4 and 5a to 5c. Again, the apparatus includes a fixation plate, in particular a lateral plate 40, located at a lateral surface of the proximal humerus 30, and a support element, in particular an intramedullary plate 50, implanted in the proximal humerus 30 (the humerus 30 is represented in a transparent form in FIGS. 5a to 5c to aid visualisation of the positioning of the intramedullary plate 50 and associated connecting and fixing screws). The plates 40, 50 are adapted to be fixed to the humerus 30 and connected to each other. Further, the axes of elongation of the lateral and intramedullary plates 40, 50 are substantially parallel to each other and to the bone axis.

While the general purpose of the apparatus is similar, the apparatus of FIGS. 4 and 5a to 5c differs from the apparatus of the preceding embodiment with regards the shape of the lateral and intramedullary plates, the arrangement of fixing and connecting screws, and the arrangement of the openings in the plates through which these screws extend.

In this embodiment, the intramedullary plate 50 has a less pronounced widening towards its proximal end 51 than the intramedullary plate 20 shown in FIG. 3. Further, the width of the intramedullary plate 50 tapers from a position 52 approximately half way along its length to its distal end 53. The intramedullary plate 50 again includes elongate openings 56a, 56b, 56c for receiving connecting screws 13. In this embodiment, each of the elongate openings 56a, 56b, 56c is elongated substantially along the axial direction of the plate, indicated by arrow 58 in FIG. 4.

Adjacent its proximal end 51, the intramedullary plate 50 is bent in a lateral direction. The bend 541 defines a proximal region 54 of the intramedullary plate 50 that corresponds to the portion of the intramedullary plate 50 that extends into the humeral head 32. The bend angle is about 10 degrees in this embodiment although it may be between 5 and 20 degrees or otherwise. An opening 56a is provided in the proximal region 54 to receive a connecting screw 13 that extends in a substantially proximal-medial direction from the lateral plate 40. The bending of the plate 50 is such as to orient the proximal region 54 so that the connecting screw 13 extends through the opening 56a in a direction that is substantially perpendicular to the surrounding portion of the plate 50, improving load distribution and stability.

In this embodiment, fixing screws 21 that fix the intramedullary plate 50 to the proximal humerus are adapted to extend through openings 57 provided either side of a distal opening 56c in the plate 50, the distal opening 56c being adapted to receive a connecting screw 13 extending from the lateral plate 40. In general, connecting screws 13 extending from the lateral plate 40 are received through openings 56a, 56b, 56c in the intramedullary plate 50 that are distributed substantially along the entire length of the intramedullary plate in this embodiment.

A plurality of distal fixing screws 11 fix a distal portion of the lateral plate 40 to the bone, and a plurality of proximal fixing screws 12 fix a proximal portion of the lateral plate 40 to the bone.

The intramedullary plate 50 can be positioned against the internal surface of the medial shaft. The lateral plate 40 can be positioned in accordance with the positioning of known lateral plates, used in standard techniques for repairing proximal humeral fractures, or otherwise. For example, the lateral plate 40 can be positioned posterior to the biceps groove and distal to the greater tuberosity of the humerus.

The configuration of the lateral plate 40 is most easily seen in FIG. 5a. The lateral plate 40 includes a plurality of substantially round openings 44 at a proximal portion 45, a plurality of substantially round openings 46 at a distal portion 47 and an elongate opening 49 substantially midway along the plate 40. The elongate opening 49 is elongated in substantially the axial direction of the lateral plate 40, which is indicated by arrow 48 in FIG. 5a.

The proximal portion 45 of the lateral plate 40 has a width that is larger than the width of the distal portion 47 of the lateral plate 40. The width of the lateral plate 40 progressively increases towards the proximal end from a central region of the lateral plate 40. The proximal portion 45 is asymmetrically arranged, such as to define a proximal-posterior wing 401. When deployed, and as can be seen in FIG. 5b, for example, the proximal-posterior wing 401 of the lateral plate 40 extends posteriorly relative to the intramedullary plate 50, and thus across a larger portion of the head 32 of the humerus than the intramedullary plate 50. The wing 401 provides for improved support in consideration of retroversion of the humeral head. When deployed, the anterior edges of the intramedullary plate 50 and the lateral plate 40 are substantially aligned.

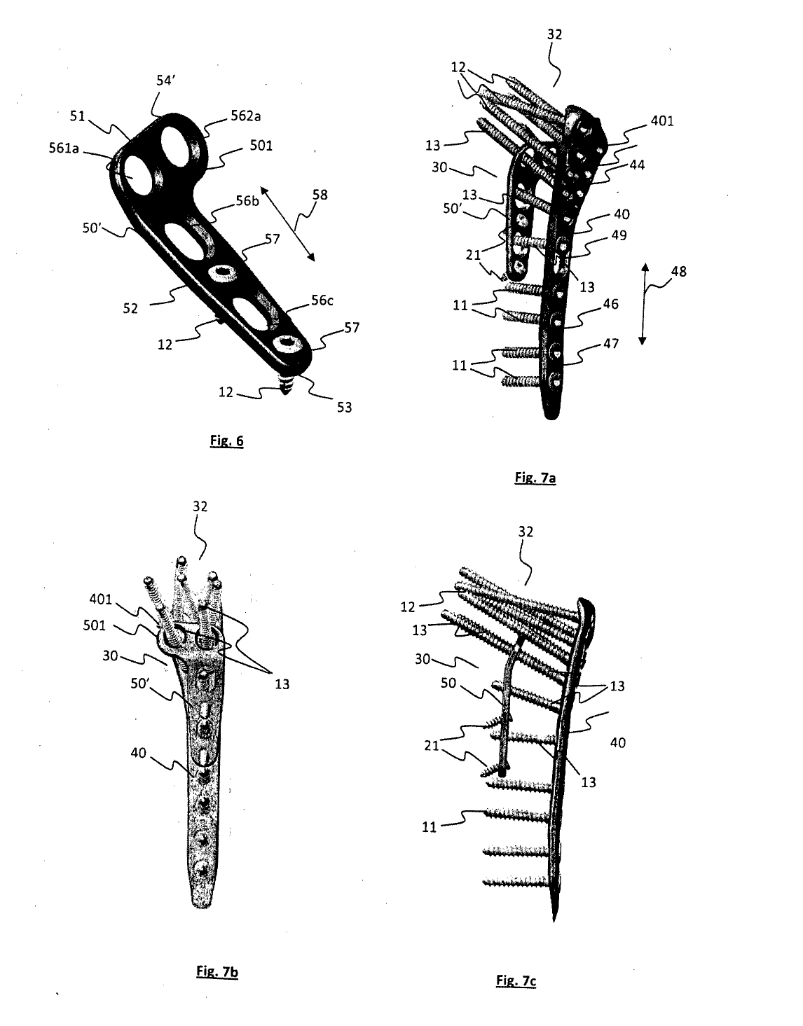

Proximal humeral fracture repair apparatus according to yet another embodiment of the present disclosure is represented in FIGS. 6 and 7a to 7c. The apparatus of this embodiment is substantially identical to the embodiment described above with reference to FIGS. 4 and 5a to 5c (with identical features being given the same reference numerals) except for the configuration of the proximal region of the intramedullary plate.

In particular, as can be seen in FIG. 6, the intramedullary plate 50′ of this embodiment has a proximal region 54′, which is again defined by a lateral bend in the plate, but which additionally comprises a proximal-posterior wing 501. The wing 501 provides the intramedullary plate 50′ with an asymmetric shape (in particular an “L”-shape in this embodiment). The proximal region 54′ has two openings 561a, 562a, one of the openings being located in the wing 501.

The proximal-posterior wing 501 at the proximal region 54′ of the intramedullary plate to some extent mirrors at least part of the proximal-posterior wing 401 of the lateral plate. Both wings 401, 501 extend posteriorly in the head 32 of the humerus to provide for enhanced support within the bone. Further, the wings 401, 501 are directly connected to each other by a connecting screw 13 that extends from the lateral plate 40 through the opening 562a in the wing 501 of the intramedullary plate 50. The wings 401, 501 can provides for improved support in consideration of retroversion of the humeral head.

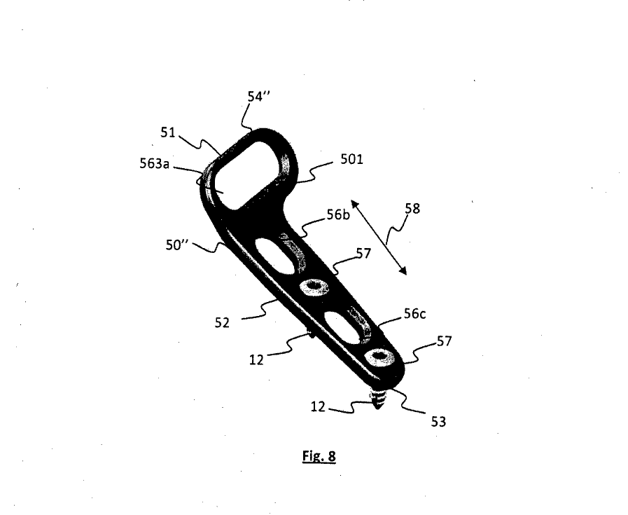

In an alternative embodiment of an intramedullary plate 50″, as shown in FIG. 8, the two openings 561a, 562b of the plate 50′ shown in FIG. 6 are replaced with a single elongate opening 563a. The elongate opening 563a extends substantially perpendicular to the axis of elongation 58 of the plate 50″. In this embodiment, rather than two connecting screws being used to connect the proximal portion 54″ of the plate 50″ to the fixation plate, a single connecting screw is used only. The elongate opening 563a provides for greater freedom in positioning of this connecting screw. Particularly when the apparatus is implanted under x-ray observation that has an anterior-posterior direction of view, the elongation of the opening 561c compensates for the difficulty of determining the angle of extension of the connecting screw in the anterior-posterior direction. The angle of extension of the connecting screw in the anterior-posterior direction, in order to extend into the opening 563a, becomes less critical due to the elongation of the opening 563a.

An intramedullary plate according to the present disclosure may have a length of about 40 to 70 mm or otherwise. For example, the intramedullary plates 50, 50′, 50″ represented in FIGS. 4, 6 and 8, respectively, have a length of about 55 mm. An intramedullary plate according to the present disclosure may have a maximum width of about 10 to 30 mm or otherwise. For example, the intramedullary plates 50, 50′, 50″ represented in FIGS. 4, 6 and 8, respectively, have a maximum width of about 14 mm and 26 mm, respectively. An intramedullary plate according to the present disclosure may have a thickness of about 1 to 5 mm or otherwise. For example, the intramedullary plates 50, 50′, 50″ represented in FIGS. 4, 6 and 8, respectively, have thickness of about 3 mm.

It will be appreciated by persons skilled in the art that numerous variations and/or modifications may be made to the above-described embodiments, without departing from the broad general scope of the present disclosure. The present embodiments are, therefore, to be considered in all respects as illustrative and not restrictive.

1. An apparatus for repair of a proximal humeral fracture comprising:
a fixation plate adapted to locate at a surface of a proximal humerus,
a support element adapted to be implanted in the proximal humerus; and
one or more connection elements to connect the fixation plate to the support element.

2. The apparatus of claim 1, wherein the support element is an intramedullary plate.

3. The apparatus of claim 1, wherein the support element is adapted to be implanted at a position where it lies against the internal surface of the medial shaft of the proximal humerus.

4. The apparatus of claim 1, wherein the fixation plate is adapted to be located at or adjacent an exterior surface of the proximal humerus, posterior to the biceps groove and distal to the greater tuberosity of the proximal humerus.

5. The apparatus of claim 1, wherein each of the fixation plate and support element have an axis of elongation and wherein, when the fixation plate and the support element are located and implanted in position, respectively, the axes align substantially parallel to each other.

6. The apparatus of claim 1, comprising a plurality of fixation elements to fix the fixation plate and/or the support element to the proximal humerus.

7. The apparatus of claim 6, wherein one or more fixation elements fix the support element to the proximal humerus and locate at positions of the support element that are distal to positions at which one or more of the connection elements connect to the support element.

8. The apparatus of claim 6, wherein one or more fixation elements fix the fixation plate to the proximal humerus and locate at positions of the fixation plate that are distal to positions at which one or more of the connection elements connect to the fixation plate.

9. The apparatus of claim 6, wherein one or more fixation elements fix the fixation plate to the proximal humerus and locate at positions of the fixation plate that are proximal to positions at which one or more of the connection elements connect to the fixation plate.

10. The apparatus of claim 6, wherein one or both of the fixation plate and the support element comprise one or more elongate openings through which the connection elements extend.

11. The apparatus of claim 10, wherein the support element comprises one or more first elongate openings through which the connection elements extend, and wherein the first elongate openings are elongated in a direction substantially perpendicular to an axis of elongation of the support element.

12. The apparatus of claim 11, wherein the first elongate openings are provided at a proximal portion of the support element.

13. The apparatus of claim 11, wherein the support element comprises one or more second elongate openings through which the fixation elements extend, wherein the second elongate openings are elongated in a direction substantially parallel to the axis of elongation of the support element.

14. The apparatus of claim 13, wherein the second elongate openings are located distally of the first elongate openings.

15. The apparatus of claim 1, wherein the fixation plate is wider at it proximal end than at is distal end.

16. The apparatus of claim 15, wherein the width of the fixation plate progressively increases towards the proximal end of the fixation plate from a central region of the fixation plate.

17. The apparatus of claim 15, wherein a proximal portion of the fixation plate is asymmetrically arranged such as to define a proximal-posterior wing of the fixation plate.

18. The apparatus according to claim 1, wherein the support element is wider at proximal portion than at a distal portion.

19. The apparatus of claim 18, wherein the width of the support element tapers towards the distal end of the support element from a central region of the support element.

20. The apparatus of claim 18, wherein the proximal portion of the support element is defined by a lateral bend in the support element.

21. The apparatus of claim 20, wherein the bend is between about 5 degrees and about 20 degrees.

22. The apparatus of claim 21, wherein the bend is about 10 degrees

23. The apparatus of any claim 18, wherein the proximal portion of the support element is asymmetrically arranged such as to define a proximal-posterior wing of the support element.

24. The apparatus of claim 17, wherein a proximal portion of the support element is asymmetrically arranged such as to define a proximal-posterior wing of the support element and wherein the wing of the support element and the wing of the fixation plate are connected by one or more connection elements.

25.-44. (canceled)

45. A method for repair of a proximal humeral fracture comprising:
implanting a support element in the proximal humerus;
locating a fixation plate at a surface of the proximal humerus; and
connecting one or more connection elements between the fixation plate and the support element.

46. (canceled)
